(19) United States
(12) Patent Application Publication
(10) Pub. No.: US 2025/0261915 A1
(43) Pub. Date: Aug. 21, 2025

(54) LINEAR CONTROL FACILITY FOR A MEDICAL FACILITY, AND MEDICAL FACILITY

(71) Applicant: Siemens Healthineers AG, Forchheim (DE)

(72) Inventor: Wolfgang NEUBER, Eschenbach (DE)

(73) Assignee: Siemens Healthineers AG, Forchheim (DE)

(21) Appl. No.: 19/055,945

(22) Filed: Feb. 18, 2025

(30) Foreign Application Priority Data
Feb. 20, 2024 (DE) 10 2024 201 520.2

(51) Int. Cl. A61B 6/04; A61N 5/10

(52) U.S. Cl. CPC A61B 6/0487; A61N 5/10; A61N 2005/1097

(57) ABSTRACT

One or more example embodiments relates to a linear control facility for a medical facility, comprising a carrier component including a fastening interface configured to mechanically couple the linear control facility to the medical facility; a carriage moveable relative to the carrier component along an adjusting axis and configured to mechanically couple a patient couch; a movement-proof drive facility fixed to the carrier component; and a spindle rotatable by the drive facility, the spindle configured to couple the carriage to the drive facility, wherein a position of the carriage relative to the carrier component along the adjusting axis is adjustable by rotating the spindle via the drive facility.

### CROSS-REFERENCE TO RELATED APPLICATION(S)

The present application claims priority under 35 U.S.C. § 119 to German Patent Application No. 10 2024 201 520.2, filed Feb. 20, 2024, the entire contents of which are incorporated herein by reference.

### FIELD

One or more example embodiments relates to a linear control facility for a medical facility. In addition, one or more example embodiments relates to a medical facility.

### RELATED ART

In the context of medical facilities it is often relevant for the examination and/or treatment of a patient to position the patient in accordance with particular requirements. Depending on the type of examination and/or treatment it may be necessary to position the patient very precisely. To this end, linear control facilities are typically used, which enable a patient positioning plate, on which the patient for example lies or sits, to be moved or positioned along an adjusting axis.

In particular in the field of radiotherapy a precise positioning of the patient is necessary in order to be able to use the medical facility to irradiate selectively only that part of the patient's tissue which does actually need to be irradiated. During the radiotherapy it may also be necessary to reposition the patient several times in order to be able to irradiate the target tissue from different directions in accordance with one or more radiotherapy treatment plans.

A lateral positioning of the patient is also of particular relevance in radiotherapy and some other types of examination and/or treatment.

In the case of the linear motors normally used at least for the lateral positioning of the patient couch, and thus of the patient positioning plate, the stator or stator track is normally firmly fixed to the medical facility, the active part being coupled to the table overlay. The latter can in this way be moved relative to the medical facility by the linear motor.

Also in the lateral direction, it is of particular relevance as regards the positioning of the patient or the patient couch that a desired target position is secured after it is reached, i.e. any unintentional change of position is prevented. Linear motors in themselves offer little or no security against an unintentional displacement along their adjusting axis. They hence have the disadvantage that they can be displaced from the target position by applying comparatively little force. To prevent this, additional and adequately dimensioned brake shoes are normally provided, which fix the patient couch in the target position by applying an additional braking force. In particular, the use of one or more friction brakes is not uncommon. Depending on the application, the brake shoes can be very large and/or complex in design, as a result of which they are comparatively expensive. This applies in particular if they are to be used to fix the patient couch against a particularly high axial force along the adjusting axis.

### SUMMARY

One or more example embodiments provide an improved concept in connection with a linear control facility for a medical facility.

### BRIEF DESCRIPTION OF THE DRAWINGS

Further advantages and details are explained below using exemplary embodiments with reference to the figures. These are schematic representations and show:

FIG. 1 an exemplary embodiment of an inventive medical facility with a functional component, an inventive linear control facility and a patient couch, and

FIG. 2 a sectional view through the inventive medical facility in FIG. 1 showing the inventive linear control facility.

### DETAILED DESCRIPTION

This is achieved in the present case by a linear control facility for a medical facility, comprising:
a carrier component having a fastening interface for the mechanical coupling of the linear control facility to the medical facility,
a carriage that can move relative to the carrier component along an adjusting axis, with a coupling means for the, in particular reversible, mechanical coupling of a patient couch to the linear control facility,
a movement-proof drive facility fixed to the carrier component and
a spindle which can be rotated by the drive facility and which couples the carriage to the drive facility,
wherein a position of the carriage relative to the carrier component along the adjusting axis can be adjusted by rotating the spindle via the drive facility.

The inventive linear control facility is designed to enable the carriage to move along the adjusting axis via the drive facility and the spindle. In this case the position of the carriage relative to the carrier component along the adjusting axis can be adjusted by corresponding actuation of the drive facility. It is preferably infinitely adjustable, so that any position along the adjusting axis which lies within an adjustment range predefined by the size of the linear control facility can be adjusted or approached by the carriage.

As far as the operating principle or method of operation of the linear control facility is concerned, the drive facility is designed to rotate the spindle and/or to brake its rotation, in particular to block it. The carriage is in turn coupled to the spindle and mounted on the carrier component, for example via a linear guide, such that a rotation of the spindle results in a movement of the carriage relative to the carrier component along the adjusting axis. The spindle consequently connects the drive facility to the carriage.

The coupling of the carriage to the spindle causes a torque of the spindle to be converted into an axial force acting on the carriage along the adjusting axis. This can be a driving force which moves the carriage, or a braking force which brakes the carriage. The carriage is preferably coupled to the spindle by one or more spindle nuts. Because of the connection geometries of the spindle and of the spindle nut(s) of the carriage, a significantly higher force is necessary compared to the linear motor described above in order to move the carriage or the active part unintentionally along the adjusting axis. Because of the coupling geometry between the spindle and the spindle nut, only a very small part of an axial force acting on the carriage along the adjusting axis is converted into a torque setting the spindle into rotation. In other words, a gear reduction of the drive facility from the carriage is preferably provided by the spindle. Moreover, the tooth flanks of the spindle nut and those of the spindle are also pressed together directly or indirectly along the adjusting axis by a large part of the axial force, as a result of which friction arises which counteracts a rotation required for the movement of the carriage. The linear control facility consequently has a significantly higher self-locking effect than a linear motor. Hence the linear control facility enables the carriage to be positioned particularly securely along the adjusting axis.

This advantage is particularly important if the linear control facility is intended to generate a linear movement in a medical facility. Here, via the fastening interface of the carrier component it can be coupled preferably firmly to the medical facility, i.e. immovable relative to the medical facility. Thanks to the coupling means a patient couch can also be coupled, in particular reversibly, to the carriage of the linear control facility. By appropriate actuation of the drive facility, the carriage with the patient couch can then be moved along the adjusting axis relative to the carrier component fixed to the medical facility. Any unintentional displacement of the patient couch along the adjusting axis is possible only with difficulty, if at all, thanks to the design of the linear control facility. Thus for instance, when positioning a patient on the patient couch, where comparatively high forces can also act on the carriage, the security against unintentional displacement of the patient couch can be increased. In general, the linear control facility in the context of medical facilities enables increased positional security when positioning the carriage and thus the patient along the adjusting axis. This applies in particular with regard to a lateral adjustment, where there is a significantly higher level of security against an unintentional change in the position of the patient couch compared to the linear drives normally used to date against impacts occurring more frequently from the side.

As far as the structure of the linear control facility is concerned, the carrier component can be understood as a base component to which the other components or elements of the linear control facility are fixed and/or mounted. The carrier component preferably has a plate-shaped design, i.e. at least substantially the shape of a plate, as a result of which it can be particularly space-saving. It is conceivable for the fastening interface to be provided at least for the most part on a first side of the carrier component, all other components of the linear control facility being fastened and/or mounted on a second side of the carrier component opposite the first side.

The fastening interface of the carrier component is preferably designed such that the carrier component can be firmly fixed to the medical facility by additional fastening means, such as screws, bolts or the like. The fastening can be effected in a form-fit and/or force-fit manner. A material-bonded connection is also conceivable, provided that non-destructive disassembly of the carrier component is not required.

The linear control facility can preferably be preassembled, the carrier component being designed to hold all other elements of the linear control facility. The carrier component can consequently be understood as a base part to which all other elements of the linear control facility are fastened. The fastening is preferably effected in each case in a form-fit and/or force-fit manner with or without corresponding fastening means. In principle, a material-bonded fastening, for example by welding, is also conceivable. As a result, it is significantly easier to handle the linear control facility as a preassembled unit before final assembly on or in the medical facility. In addition, it is possible to separate preassembly and final assembly, as a result of which the process can be improved in production, in particular to increase efficiency.

The carriage can be supported by the carrier component. However, it is not permanently positioned relative to the carrier component, but can move along the adjusting axis. This is possible for example thanks to a linear guide. To this end the linear control facility preferably has at least one guide rail, to which the carriage is displaceably coupled for guidance along the adjusting axis. As a result, a particularly precise and secure guidance of the carriage is possible. The at least one guide rail is preferably firmly fixed to the carrier component, for example by a screw connection. It is also conceivable for the carrier component to already be designed such that it has a cantilever forming the at least one guide rail.

The carriage can in principle be coupled to the at least one guide rail by any conceivable bearing. A plain bearing means is preferably provided to this end, since this is particularly inexpensive as well as low-wear and low-noise. In one or more example embodiments, the bearing means is a linear guide (e.g., profile rail guide). It is conceivable for the at least one guide rail to have a depression running along the adjusting axis on one or on two opposite sides, into which the carriage, in particular a plain bearing means of the carriage, engages. The at least one guide rail can thus be encompassed by the carriage or its plain bearing means, as a result of which it is secured against “falling off” the at least one guide rail.

It is conceivable for the carriage to have a bearing means on a first side, for example protrusions encompassing the guide rail, via which it is coupled to the at least one guide rail, and on a second side opposite the first side to have the coupling means for the, in particular reversible, mechanical coupling of the patient couch. This can be designed to couple the patient couch to the carriage, in particular without additional fastening means, and to decouple it non-destructively from the carriage again. The coupling means preferable enables fast and simple coupling and decoupling of the patient couch to or from the carriage. The patient couch can however also be firmly fastened to the carriage by the coupling means, for instance using a screw connection.

The fastening interface and/or the coupling means can enable not only the mechanical coupling but also an electrical coupling. In other words, they can be designed to couple the linear control facility to the medical facility and/or the patient couch not only mechanically, but also for the transmission of data and/or for energization. The electrical coupling can in particular take place synchronously with, i.e. at the same time as, the mechanical coupling.

The drive facility and the spindle are firmly fixed to the carrier component. Corresponding fastening means, for example screws, bolts or the like, can be provided for the fastening in each case. Any form-fit or material-bonded fastening is also conceivable. However, whereas the drive facility is fixed in relation to the carrier component, the spindle is mounted on the carrier component so that it can rotate along its longitudinal axis. To this end, the spindle is fastened to the carrier component by a bracket. The bracket preferably has one or more bearings, for example rolling bearings. It is advantageously designed so that it blocks a translational motion of the spindle relative to the carrier component, whereas a rotation of the spindle about its longitudinal axis is possible.

The spindle is particularly preferably a threaded spindle, in particular a recirculating ball screw. By appropriately selecting a thread pitch or a pitch of the tooth flanks of the spindle and of the spindle nut(s), a gear reduction can be achieved so that only a very small part of a force acting unintentionally on the carriage along the adjusting axis is converted into a rotation of the spindle which is required for the movement of the carriage. Because of the toothing, a large part of the unintentionally acting force causes the tooth flanks of the spindle nut(s) to be pressed directly or via at least one intermediate part, in the case of the recirculating ball screw the balls, along the adjusting axis onto the tooth flanks of the spindle. As a result, the friction between the tooth flanks or between the tooth flanks and the balls is increased, which additionally counteracts a rotation of the spindle.

In principle any type of threaded spindle can be used as a spindle, i.e. for example including a trapezoidal threaded spindle or a rolling threaded spindle. However the form of embodiment of the recirculating ball screw is preferred, since this is comparatively inexpensive and low-maintenance. Moreover, it enables particularly low-noise operation.

In one preferred form of embodiment the drive facility has a motor with a rotor shaft for rotating the spindle, and a break engaging on the rotor shaft for braking and/or locking the rotation of the spindle. The rotor shaft can be set in rotation by the motor and can be braked and/or locked by the brake. Starting from it, a torque, in particular the driving and braking torque, can be transmitted directly or indirectly to the spindle. The driving torque and the braking torque counteract each other.

The motor is preferably an electric motor, which comprises not only the rotor shaft but also a rotor and a stator connected thereto. The brake is preferably a friction brake which engages on the rotor shaft. The drive facility can also have several such brakes. In addition, the motor, providing it is an electric motor, can also have a motor brake, via which a deceleration of the rotational movement of the rotor shaft is achieved by generating a magnetic field which counteracts the rotation of the rotor shaft. Overall, the drive facility is thus preferably designed to apply a driving torque and a braking torque to the rotor shaft. The drive facility is preferably fixed as a unit to the carrier component using a suitable bracket.

The rotor shaft can in principle be directly connected to the spindle. In one form of embodiment it can also be a part or section of the spindle, so that the motor and the brake engage directly on the spindle. However, it is preferable for the drive facility to have a torque transmission facility which is designed to transmit the driving torque and/or the braking torque of the rotor shaft to the spindle, wherein the torque transmission facility comprises a belt drive, in particular with a toothed belt, and/or a gear unit. The torque transmission facility consequently serves to couple the rotor shaft to the spindle. Thus the spindle on the one hand and the motor with the brake or the drive facility on the other hand can each be provided as separate components, as a result of which the manufacture and maintenance of the linear control facility can be simplified.

It is conceivable for the rotor shaft of the motor to be coupled to the spindle by the torque transmission facility without a gear unit. In this case a toothed wheel can be non-rotatably connected to the rotor shaft and a second toothed wheel can be non-rotatably connected to the spindle, the spindle and the rotor shaft being coupled to one another by a belt, in particular a toothed belt, engaging in both toothed wheels.

However, the torque transmission facility preferably has a gear unit and a toothed belt. The gear unit can in this case be interposed between the rotor shaft of the motor and a drive shaft of the torque transmission facility, so that the driving torque can be applied to the drive shaft by the rotor shaft via the gear unit. The spindle and the drive shaft can each have a toothed wheel, the toothed belt engaging in both the toothed wheels. Thus a first transmission ratio, preferably a gear reduction, of the drive torque and/or braking torque can be created between the rotor shaft and the drive shaft. A second transmission ratio, preferably a gear reduction, can be created downstream by the belt drive, thanks to which the driving or braking torque acting on the spindle can be adjusted once again. In other words, the torque transmission facility for transmitting torque from the rotor shaft to the spindle preferably has a gear unit stage provided by the gear unit and a belt stage provided by the toothed belt.

Instead of the toothed belt, the use of a V-belt or a chain or the like is also conceivable. However, compared to the V-belt the toothed belt offers the advantage that a greater torque can be transmitted with it, it being possible to prevent “slippage” of the toothed belt thanks to the toothing. Compared to a chain, the toothed belt moreover enables a significantly lower-noise torque transmission. Furthermore, the cost factor and the freedom from maintenance also justify the use of a toothed belt.

Thanks to the spindle and/or the torque transmission facility a gear reduction between the rotor shaft and the carriage can thus be predefined or adjusted. This gear reduction enables an increase in the braking torque that can be generated by the brake, so that ultimately a higher braking force can be applied to the carriage of the linear control facility and as a result it can be better secured against any unintentional displacement. As explained in the introduction, thanks to the spindle pitch there is already a gear reduction between the spindle and the carriage. This can advantageously be supplemented, i.e. increased, by one or more gear reductions of the torque transmission facility.

In order to achieve the greatest possible gear reduction via the torque transmission facility, it is preferable for the torque transmission facility to have a gear unit and a toothed belt. Thanks to the gear unit stage, which can for example be provided between the rotor shaft and the drive shaft, a first gear reduction of the braking torque can be achieved. Thanks to the belt stage of the toothed belt, in particular downstream of the gear unit stage, a second gear reduction of the braking torque can additionally take place, before finally the geared-down and thus significantly increased braking torque is applied to the spindle. The driving or braking torque applied to the spindle in each case can thus be increased by the torque transmission facility compared to the driving or braking torque applied to the rotor shaft in each case. Thanks to the further (third) gear reduction between the spindle and the carriage the braking force generated by the braking torque of the spindle on the carriage can be further increased.

The multiple gear reduction has the advantage that a very high braking force or holding force along the adjusting axis can be exerted on the carriage, in particular even with a brake which is comparatively small. This represents a significant advantage compared to the linear motors described in the introduction and the brakes required in this context. Because of the “increase” of the braking torque or the braking force thanks to the gear reduction, in accordance with one or more example embodiments a smaller brake is sufficient to decelerate and/or lock the carriage. Expensive (large) brake shoes, as required for linear motors, can thus be dispensed with in accordance with one or more example embodiments.

The gear reduction can be fixed in accordance with the selected components. Alternatively, it can be adjustable, for example by adjusting a size or numbers of teeth of toothed wheels of the drive shaft and of the spindle or the like. Even if this is preferred, the torque transmission facility need not in principle have an additional gear unit, so that for example the rotor shaft can be coupled to the spindle by a toothed belt. To this end, both shafts can have a corresponding toothed wheel, non-rotatably fastened to the respective shaft, into which the toothed belt can engage. In this case only a gear reduction between the rotor shaft and the spindle by the torque transmission facility and between the spindle and the carriage is then possible.

Particularly preferably, the linear control facility is designed such that a force for moving and/or braking, in particular locking, the carriage can be transmitted to the carriage by the drive facility substantially slippage-free. In other words, the entire drive train of the linear control facility is preferably designed at least substantially as completely form-fit, so that there can be no slippage between the individual components. As a result, the positioning accuracy of the carriage along the adjusting axis can be improved. The carriage is thus better able to approach a target position. Especially in dynamic applications, in which the position of the carriage along the adjusting axis is changed quickly, in particular when changing direction, the positioning accuracy thus achieved is of advantage.

The freedom from slippage requires that all elements of the drive train are correspondingly designed and connected to one another. In this case it is of advantage if the torque transmission facility has a toothed belt. This can be made of rubber and pretensioned, so that the rotor shaft or, where present, the drive shaft is coupled to the spindle slippage-free. In this case the elasticity of the toothed belt can be utilized. A suitable belt tensioner can be provided to tension the toothed belt. In addition, thanks to the previously described use of a recirculating ball screw and one or more spindle nuts it is possible to prevent slippage in the drive train even when coupling the carriage to the spindle. This advantage can in principle be achieved by any type of threaded spindle.

Additionally, to prevent slippage it is advantageous if the components of the drive train and their fastening to the carrier component have a high rigidity. The carrier component, the brackets of the drive facility and of the spindle, the carriage and/or the at least one guide rail are hence preferably made of metal, in particular stainless steel or aluminum. The spindle is preferably made of stainless steel, in particular machine steel. It, and also the at least one guide rail, is preferably a standard part, so that costs can be saved.

For the most precise positioning possible of the carriage along the adjusting axis the linear control facility preferably has a measuring facility which comprises a drive measuring means (a drive measurer) designed to measure an angle of rotation of the rotor shaft of the drive facility. The drive measuring means can be a resolver or can comprise such a resolver. Additionally or alternatively, the drive measuring means can be an encoder or can comprise such an encoder. Thanks to the drive measuring means the angle of rotation of the rotor shaft can thus be determined. Alternatively or additionally it is conceivable for the drive measuring means, providing the torque transmission facility has a drive shaft as an intermediate stage, to be designed to measure an angle of rotation of the drive shaft. If the transmission ratio or the gear reduction ratio of the drive train is known by the rotor shaft and/or the drive shaft as far as the carriage, the position of the carriage along the adjusting axis can be calculated using the angle of rotation of the rotor shaft and/or drive shaft. It is conceivable for the drive measuring means to be an incremental encoder or an absolute encoder or to comprise such an incremental encoder or absolute encoder. It can be assembled as part of the drive facility or separately on the carrier component.

Additionally or alternatively, the measuring facility can comprise a carriage measuring means designed to measure a position and/or a distance traveled of the carriage along the adjusting axis. This can be a measuring means for detecting an absolute position or a relative position of the carriage along the adjusting axis. In one or more example embodiments, the carriage measuring means is a magnetic linear measurement system with a magnetic tape with a read head. In the latter case, a starting point is necessary to determine the actual position of the carriage along the adjusting axis, from which the actual absolute position of the carriage can be ascertained using the determined relative position. To this end, at least one position sensor, in particular a light barrier, can be provided. The position sensor can be mounted on the carrier component, on the carriage or on the at least one guide rail. It can be approached by appropriate actuation of the drive facility at the beginning of operation of the linear control facility, in particular once, in order to calibrate the carriage measuring means.

The measuring facility preferably has a drive measuring means and a carriage measuring means. Thanks to the resulting redundancy the fail-safe nature of the measuring facility can be improved. This is in particular of advantage if a particularly precise positioning of the carriage along the adjusting axis is necessary, using the linear control facility. To this end, the linear control facility can have a control facility, via which it is designed to compare measurement results of the drive measuring means with measurement results of the carriage measuring means and as a function of the comparison to assess the operability of the measuring facility and/or of the linear control facility. The internal control facility can be mounted on the carrier component. Additionally or alternatively, the linear control facility can be designed to carry out, via an external control facility, a corresponding comparison of the measurement results and a corresponding assessment of the operability of the measuring facility and/or of the linear control facility. In this case the linear control facility preferably has a suitable interface, via which it can be coupled to the external control facility. Such an external control facility can for example be a central computer of a medical facility.

“Measurement results” can be understood as the measurement data determined by the drive measuring means and carriage measuring means, i.e. in the case of the drive measuring means the angle of rotation of the rotor shaft and/or of the drive shaft and in the case of the carriage measuring means the position and/or the distance traveled by the carriage along the adjusting axis. It is also conceivable for each “measurement result” to relate to a probable position of the carriage along the adjusting axis determined on the basis of this data, i.e. a position at which the carriage should be located assuming that the data has been captured correctly.

The control facility, regardless of whether it is part of the linear control facility or is connected as an external control facility to the linear control facility, enables a comparison of the measurement results of both measuring means. If the measurement results match, the actual position of the carriage can be determined on the basis of the measurement results. If on the other hand they do not match, the presence of an operating error in the measuring facility and/or in the linear control facility can be identified. It is hence of advantage if the control facility is additionally designed to actuate the drive facility. Thus the operation of the linear control facility can be stopped automatically when an operating error is detected, in particular by outputting a warning. Such a warning can for example be output as a visual and/or acoustic signal on the medical facility and/or on an operating system for, in particular external, operation of the medical facility and/or a patient couch coupled to the linear control facility. The control facility can also then be designed to output a warning if it is not designed to actuate the drive facility.

As mentioned, the linear control facility can preferably have at least one control interface, which is designed for electrical power transmission and/or for the transmission of data between the linear control facility and the medical facility or between the linear control facility and the patient couch. The linear control facility, in particular the drive facility and/or the measuring facility and/or the control facility, can thus be energized by the medical facility. Additionally or alternatively, an exchange of data, in particular the aforementioned measurement results, is possible between the linear control facility and the medical facility and/or between the linear control facility and the patient couch. It is conceivable in this case for the drive facility of the linear control facility to be controlled by a control panel and/or a central computer of the medical facility. It is also conceivable for the drive facility of the linear control facility to be controlled by a control panel of the patient couch.

Besides the inventive linear control facility one or more example embodiments also relates to a medical facility for the medical examination and/or treatment of patients, comprising a functional component and at least one inventive linear control facility. All features, explanations and advantages described in connection with the inventive linear control facility can be transferred to the inventive medical facility, also including the functional component, and vice versa.

The functional component can be any component which is designed to achieve the purpose intended by the medical facility. A gantry or any component designed for medical imaging and/or irradiation are for example conceivable. The functional component can preferably be a component which comprises a beam generator for generating radiation, in particular photon and/or electron radiation of high beam intensity.

In particular, the inventive linear control facility is, as described in the introduction, coupled to the functional component of the medical facility via the fastening interface. It is for instance conceivable for the carrier component of the linear control facility to have suitable drill holes, at which it can be screwed on or in the functional component.

It is further preferable for the medical facility to comprise a patient couch for positioning a patient, wherein the patient couch is or can be coupled to the carriage of the linear control facility by the coupling means. The patient couch can thus be coupled either permanently or reversibly to the carriage of the linear control facility by the coupling means. It is conceivable for the patient couch to be a mobile patient couch which prior to the examination and/or treatment is coupled to the carriage of the linear control facility and after the examination and/or treatment is decoupled from the carriage again. To this end, the carriage and the patient couch can have a suitable, in particular magnetic and/or (electro-) mechanical, coupling.

The patient couch can preferably be moved along the adjusting axis via the linear control facility, the adjusting axis being oriented perpendicular to a longitudinal axis of the patient couch. The longitudinal axis of the patient couch is to be understood as the axis along which the patient couch has its greatest extent. It is normal for patients to be pushed into and out of the medical facility along this longitudinal axis during the examination and/or treatment with a medical facility, for example a magnetic resonance facility. If the adjusting axis of the linear control facility is oriented, in particular in the horizontal direction, perpendicular to the longitudinal axis of the patient couch, then in addition to the forward and backward movement along the longitudinal axis, a lateral movement of the patient couch to the left and right is also possible, as a result of which more freedom of movement is provided.

The medical facility is preferably a facility for the medical irradiation of a patient, in particular a radiotherapy facility. In this case the functional component can, as explained above, preferably comprise a beam generator designed to generate radiation, in particular photon and/or electron radiation of high beam intensity. For example, the functional component can be or comprise a gantry, in which the beam generator is movably guided. In medical irradiation or radiotherapy, the precise and improved positioning of the patient couch, which is made possible by the inventive linear control facility and is secured against unintentional displacement, is of particular advantage. Here it is not uncommon for it to be necessary to position the patient couch at least once transversely to the longitudinal axis of the patient couch, i.e. laterally or sideways, during the treatment of the patient, for instance to be able to irradiate a particular, non-central part of the tissue of the patient.

Normally a central computer of the radiotherapy facility calculates radiotherapy treatment plans which relate to the position of the patient as regards the target tissue. The central computer can be provided as part of the functional component. The linear control facility is hence coupled, preferably via a control interface, to the functional component, in particular its central computer. The drive facility of the linear control facility can then be actuated by the central computer in accordance with the radiotherapy treatment plan(s). Thus the at least one position of the patient couch specified by the one or more radiotherapy treatment plans can be approached.

In principle, the installation of the inventive linear control facility is also conceivable in any other medical facility in which a corresponding positioning of a patient couch perpendicular to its longitudinal axis is necessary or advantageous.

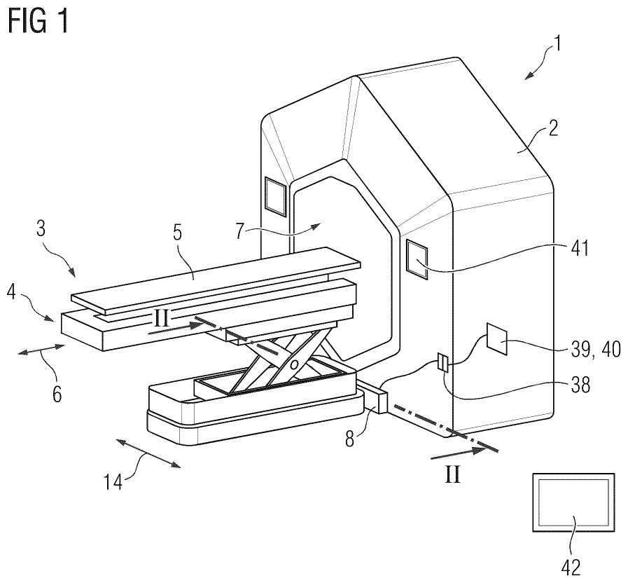

FIG. 1 shows an exemplary embodiment of an inventive medical facility 1 for the examination and treatment of a patient. In the present case the medical facility 1 is a radiotherapy facility. It comprises a gantry 2 as a functional component. In other forms of embodiment, instead of the gantry 2 any other functional component is conceivable which is designed to achieve the (treatment) objective or the purpose of the medical facility 1, i.e. for instance imaging or radiotherapy or similar. Besides the gantry 2 the medical facility 1 comprises a patient couch 3 with a table overlay plate 4 and a supporting surface 5 for positioning a patient. The patient couch 3 is designed to move the table overlay plate 4 along a longitudinal axis 6 of the patient couch 3. Thus the table overlay plate 4 can be moved (with a patient) into and out of the tube 7.

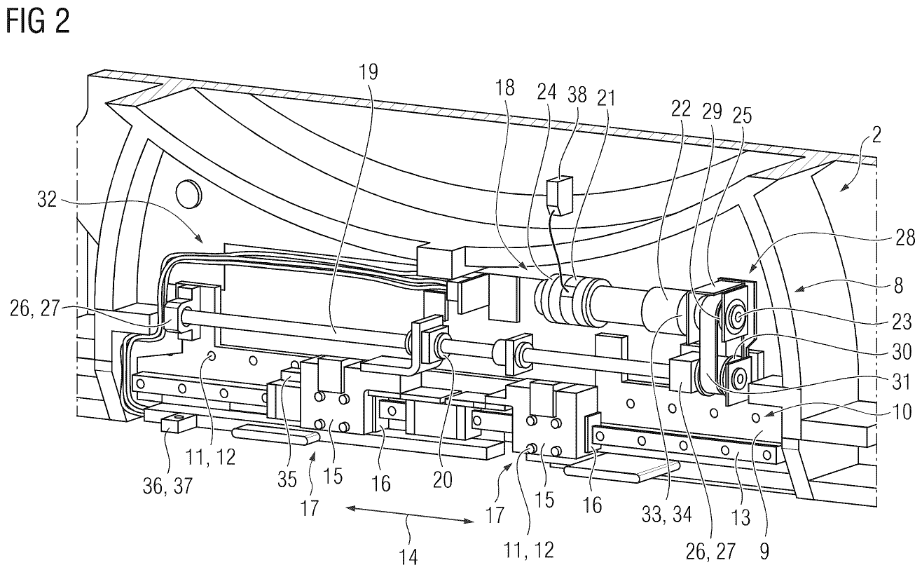

The patient couch 3 is coupled to the functional component or gantry 2 via an exemplary embodiment of an inventive linear control facility 8 of the medical facility 1. To clarify the structure and method of operation of the linear control facility 8 FIG. 2 shows a sectional view through the medical facility 1 in FIG. 1 showing the linear control facility 8.

On the basis of FIG. 2 it becomes clear that the linear control facility 8 has a plate-like carrier component 9 with a fastening interface 10. The carrier component 9, and thus the linear control facility 8, is in this case fixed to the gantry 2 via several drill holes 11 and screws 12 forming the fastening interface 10. The linear control facility 8 moreover has a guide rail 13 which is firmly screwed to the carrier component 9 and runs along an adjusting axis 14. A carriage 15 is displaceably mounted on it by a sliding bearing 16 or a sliding bearing means 16 relative to the carrier component 9 along the adjusting axis 14, so that the carriage 15 can be moved along the adjusting axis 14 relative to the gantry 2. The carriage 15 in turn has a coupling means 17, which in the present case comprises several drill holes 11 and screws 12 and by which the patient couch 3 is coupled to the carriage 15 (in FIG. 2 the patient couch 3 is “cut off” in the region of the coupling).

The linear control facility 8 thus represents the link between the gantry 2 and the patient couch 3. Thanks to the linear control facility 8 the position of the patient couch 3 can be adjusted in the lateral direction along the adjusting axis 14 oriented perpendicular to the longitudinal axis 6. To this end, the linear control facility 8 has a drive facility 18, by which a spindle 19, in the present case a recirculating ball screw, can be rotated. The carriage 15 is coupled to the spindle 19 via a spindle nut 20, such that its position along the adjusting axis 14 can be adjusted continuously by the rotation of the spindle 19. In this case the carriage 15 is guided on the guide rail 13.

Both the drive facility 18 and the spindle 19 are fastened to the carrier component 9 via respective brackets 25, 26. The brackets 26 of the spindle 19 each comprise a rolling bearing 27, by which the spindle 19 is fastened to the carrier component 9 so as to be rotatable about its longitudinal axis.

The drive facility 18 in the present case has an electric motor 21 with a rotor shaft. This is coupled to a drive shaft 23 via a gear unit 22 of a torque transmission facility 28. Furthermore, the drive facility 18 comprises a brake 24, in the present case a friction brake, which engages on the rotor shaft of the electric motor 21 and is designed to apply a braking torque to the rotor shaft and also, via the gear unit 22, to the drive shaft 23, in order thereby to decelerate or lock the movement of the carriage 15 along the adjusting axis 14. Thanks to a gear reduction of the gear unit 22 between the rotor shaft and the drive shaft 23 the braking torque generated with the brake 24 is increased on the drive shaft 23.

The driving torque and the braking torque of the rotor shaft is transmitted to the spindle 19 by the torque transmission facility 28. In the present case the torque transmission facility 28 comprises not only the gear unit 22 but also a toothed wheel 29 connected non-rotatably to the drive shaft 23, a toothed wheel 30 connected non-rotatably to the spindle 19, and a toothed belt 31, the teeth of which engage in the teeth of both the toothed wheels 29, 30. The toothed wheels 29 and 30 are selected so that a further gear reduction is present between the drive shaft 23 and the spindle 19, by which the braking torque of the drive shaft 23 occurring at the spindle 19 is further increased.

Finally, the geometry of the spindle 19 and of the spindle nut 20 causes a further gear reduction of the braking torque acting on the carriage 15 through the spindle 19 or of the braking force acting on the carriage. Overall, the braking torque that can be applied to the rotor shaft of the electric motor 21 by the brake 24 is thus reduced several times along the drive train of the linear control facility 8. As a result, a very high holding force can be exerted on the carriage 15 by the brake 24, making it impossible or very difficult for it to be displaced along the adjusting axis 14. Because of the gear reduction of the braking torque of the brake 24 resulting from the structure of the drive train, the position of the patient couch 3 along the adjusting axis 14 is particularly well secured against unintentional displacement. In particular in comparison with conventional linear motors, the linear control facility 8 offers significantly more security, it being at the same time possible to dispense with large, costly brake shoes.

It is also possible to dispense with the drive shaft 23 and the gear unit 22 in other embodiments. The toothed wheel 29 can then be fastened directly to the rotor shaft of the electric motor 21, so that the rotor shaft is coupled directly to the spindle 19 via the toothed belt 31. However, this can have an impact on the gear reduction achievable with the drive train.

Some components of the linear control facility 8 are provided as standard components, in particular as standard parts, meaning that production is simple and inexpensive. Specifically, in the present case the spindle 19, the drive facility 18, the toothed belt 31, the guide rail 13 and the bearings 16, 17 are provided as standard components or purchased parts.

The linear control facility 8 further offers the property and the advantage that the force for moving and braking, in particular locking, the carriage 15 along the adjusting axis 14 can be transferred from the drive facility 18, specifically the rotor shaft, to the carriage 15 at least substantially slippage-free. As a result, it is possible to position the carriage 15, and thus the patient couch 3, along the adjusting axis 14 particularly exactly. This is of advantage especially in dynamic applications with a high speed of movement of the carriage 15.

To this end, the drive train of the linear control facility 8 is configured accordingly. In detail, the carrier component 8 and the brackets 25, 26 of the drive facility 18 and of the spindle 19 are designed to be particularly rigid. The carriage 15 is also guided on the guide rail 13 at least substantially free of play. Thanks to the use of the V-belt 31 for torque transmission, slippage can be at least substantially prevented even while coupling the rotor shaft or the drive shaft 23 to the spindle 19. The same applies for coupling the carriage to the recirculating ball screw 19 via the spindle nut 20.

In order to further improve the positional accuracy of the carriage 15 along the adjusting axis 14, the linear control facility 8 has a measuring facility 32. On the one hand, this comprises a drive measuring means 33, in this case a resolver 34 or rotary encoder, which is designed to measure an angle of rotation of the drive shaft 23. However, an encoder would also be conceivable as the drive measuring means 33. The resolver 34 outputs the current angle of rotation of the rotor shaft as the measurement result. In other embodiments, it would additionally or alternatively be conceivable for the drive measuring means 33 to be designed to measure an angle of rotation of the drive shaft 23 of the electric motor 21.

In addition, the measuring facility 32 comprises a carriage measuring means 35, which is designed to output a distance traveled of the carriage 15 along the adjusting axis 14 as a measurement result. The measuring facility 32 further comprises a position sensor 36, in this case a light barrier 37, by which a zero position or zero location of the carriage 15 along the adjusting axis 14 can be determined.

To actuate the drive facility 18 and to assess the measurement results of the measuring facility 32, in particular of the drive measuring means 33 and of the carriage measuring means 35, the linear control facility has a control interface 38, via which it is connected to the gantry 2, specifically to a control facility 39 of the gantry 2, in this case a central computer 40. At the beginning of operation of the linear control facility 8, the carriage 15 is moved once over the light barrier 37 by the control facility 39 in order to calibrate the measuring facility 32 and to determine the zero location of the carriage 15 along the adjusting axis 14. The control facility 39 is designed to compare the measurement results of the resolver 34 with those of the carriage measuring means 35 during the operation of the linear control facility 8. If these match, operation of the linear control facility 8 is continued. However, if they do not match, operation is automatically interrupted with the output of a visual message on a control panel 41 of the gantry 2 and an external computer 42, which is designed to control and monitor the medical facility 1. An example of a corresponding message is: “Error in the lateral adjustment of the patient couch”. The output of an acoustic signal is also conceivable.

The linear control facility 8 is moreover energized by the gantry 2 via the control interface 38.

It should further be stressed that the carrier component 9 is designed to support all other components and elements of the linear control facility 8. Thus the linear control facility 8 can be preassembled as a module, before it is mounted on the gantry 2 later on via the fastening interface 10. This in particular has logistical and production-related advantages.

The inventive linear control facility can be provided generally in any medical facility, in particular in those medical facilities in which a precise linear movement, for instance of a patient couch, and/or high security of the patient couch against an unintentional displacement is preferable. Use in a magnetic resonance facility or a computed tomography facility is for instance conceivable.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative used descriptors herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the invention has been illustrated and described in greater detail by the preferred exemplary embodiment, the invention is nevertheless not restricted by the disclosed examples and other variations can be derived therefrom by the person skilled in the art, without departing from the scope of protection of the invention.

Independent of the grammatical term usage, individuals with male, female or other gender identities are included within the term.

1. A linear control facility for a medical facility, comprising:
a carrier component including a fastening interface configured to mechanically couple the linear control facility to the medical facility;
a carriage moveable relative to the carrier component along an adjusting axis and configured to mechanically couple a patient couch;
a movement-proof drive facility fixed to the carrier component; and
a spindle rotatable by the drive facility, the spindle configured to couple the carriage to the drive facility,
wherein a position of the carriage relative to the carrier component along the adjusting axis is adjustable by rotating the spindle via the drive facility.

2. The linear control facility of claim 1, wherein the spindle is a threaded spindle.

3. The linear control facility of claim 1, further comprising:
at least one guide rail, the carriage being displaceably coupled to the least one guide rail for guidance along the adjusting axis.

4. The linear control facility of claim 1, wherein the drive facility includes a motor, a rotor shaft to rotate the spindle and a brake configured to engage the rotor shaft to at least one of brake or lock the rotation of the spindle.

5. The linear control facility of claim 4, wherein
the drive facility includes a torque transmission facility configured to transmit at least one of a driving torque or a braking torque of the rotor shaft to the spindle, and
the torque transmission facility includes at least one of a belt drive or a gear unit.

6. The linear control facility of claim 4, wherein a gear reduction between the rotor shaft and the carriage is predefined or adjustable.

7. The linear control facility of claim 1, wherein the linear control facility is configured to transmit a force for at least one of moving or braking the carriage from the drive facility to the carriage at least substantially slippage-free.

8. The linear control facility of claim 4, further comprising:
a measuring facility, wherein the measuring facility includes at least one of
a drive measurer configured to measure an angle of rotation of the rotor shaft of the drive facility, or
a carriage measuring means configured to measure at least one of a position of the carriage or a distance traveled of the carriage along the adjusting axis.

9. The linear control facility of claim 8, wherein the linear control facility is configured to compare, via at least one of a control facility of the linear control facility or an external control facility, measurement results of the drive measurer with measurement results of the carriage measuring means and is configured to assess an operability of at least one of the measuring facility or the linear control facility as a function of the comparison.

10. The linear control facility of claim 1, further comprising:
at least one control interface configured to control at least one of electrical power transmission or transmission of data between the linear control facility and the medical facility or between the linear control facility and the patient couch.

11. The linear control facility of claim 1, wherein
at least a portion of the linear control facility is preassembled, and
the carrier component configured to hold all other elements of the linear control facility.

12. A medical facility for at least one of medical examination or treatment of patients, the medical facility comprising:
a functional component; and
at least one of the linear control facility of claim 1, wherein the linear control facility is coupled to the functional component via the fastening interface.

13. The medical facility of claim 12, wherein the medical facility comprises a patient couch for positioning a patient, and the patient couch is at least one of coupled or coupleable to the carriage of the linear control facility.

14. The medical facility of claim 13, wherein the patient couch is moveable via the linear control facility along the adjusting axis, the adjusting axis being oriented perpendicular to a longitudinal axis of the patient couch.

15. The medical facility of claim 12, wherein the medical facility is a facility for medical irradiation of a patient.

16. The linear control facility of claim 2, wherein the spindle is a recirculating ball screw.

17. The linear control facility of claim 5, wherein the torque transmission facility includes the belt drive and the belt drive includes a toothed belt.

18. The linear control facility of claim 8, wherein the drive measurer is a resolver.

19. The medical facility of claim 15, wherein the medical facility is a radiotherapy facility.
